(19) United States
(12) Patent Application Publication
(10) Pub. No.: US 2010/0021572 A1
(43) Pub. Date: Jan. 28, 2010

(54) Nutritional Drink

(76) Inventor: Mitchell Karl, Boca Raton, FL (US)

Correspondence Address: James David Johnson, P.A., 401 East Las Olas Boulevard, Suite 130-290, Fort Lauderdale, FL, 33301, US

(21) Appl. No.: 12/576,035

(22) Filed: Oct. 8, 2009

Related U.S. Application Data
(63) Continuation-in-part of application No. 12/120,765, filed on May 15, 2008.
(60) Provisional application No. 60/942,122, filed on Jun. 5, 2007.

(51) Int. Cl. A61K 36/899; A61P 7/00

(52) U.S. Cl. 424/750

(57) ABSTRACT

A first composition of drink ingredients specifically designed to lower cholesterol that addresses multiple mechanisms including hepatic synthesis and release, intestinal absorption of cholesterol, while, at the same time, including ingredients that mitigate the side effects of the constituents and increase their efficacy by affecting emotional factors that influence compliance such as a sense of well-being and euphoria on the one hand, or an increased overall metabolism and desire for the product stemming from its coloration on the other hand.

### CROSS-REFERENCE TO RELATED APPLICATION

This application is a continuation-in-part application of U.S. nonprovisional patent application Ser. No. 12/120,765 filed on May 15, 2008, which claims the priority of U.S. provisional patent application Ser. No. 60/942,122 filed on Jun. 5, 2007. Both applications are herein incorporated by reference in their entirety.

### FIELD OF THE INVENTION

This invention relates to nutritional supplements. More particularly, the invention relates to compositions and methods for supplementing the diet for improving health and preventing disease.

### BACKGROUND

Chronically elevated blood levels of cholesterol lead to cardiovascular disease as the cholesterol finds its way into the walls of blood vessels and damages them. This ultimately results in symptoms of chronic arterial insufficiency such as angina and claudication on the one hand, and acute vascular insufficiency, such as heart attack and stroke on the other.

Over $120 billion dollars is spent on direct and indirect costs associated with cardiovascular disease annually in the United States alone. Cardiovascular disease incidence increases with serum LDL cholesterol in a log linear fashion and more importantly declines with treatment-induced reduction of serum LDL cholesterol.

Conventional therapy for elevated blood cholesterol levels takes the form of four classes of FDA approved medications: statins, bioresins, fibrates, and niacin.

Of these, statins are the most widely used with greater than $20 billion in annual sales; however, all the classes, including statins, have side effects and at higher doses, that are necessary to achieve targets, result in side effects that limit their utility. This is especially so of niacin and statins

Recently, it has been appreciated that in attempting to lower cholesterol, two or more drugs with different mechanisms of action can lower toxicity and produce synergy in the cholesterol lowering effect. Vytorin, a recently introduced combination of Zetia and Simvastatin, has been shown to decrease cholesterol absorption and synthesis and reduce cholesterol better than the sum of the expected reduction of either drug alone. This is explained by a phenomenon I refer to as “escape homeostasis.” When one pathway to cholesterol elevation is blocked, an alternative pathway is often enhanced by the body, so that the overall cholesterol levels are maintained. There is, thus, a built-in or automatic drug resistance that can only be overcome with multiple active agents working simultaneous at different sites. It is noteworthy that evidence exists that even homeopathic, previously felt sub-therapeutic amounts of biologically active cholesterol lowering compounds can exert powerful efficacy with minimal side effects when combined with other agents that work by alternative pathways.

### SUMMARY

The invention is based on the development of a cholesterol lowering beverage that utilizes multiple mechanisms and sites of action to reduce cholesterol and also has ingredients that reduce toxicity, increase the rate of metabolism both on a general and a cellular level and, finally stimulate compliance by causing a sense mild euphoria and well-being, as well as enhancing metabolism.

Accordingly, in one aspect, the invention features a nutritional beverage that includes water and at least two active ingredients selected from among the following: niacin, a phytosterol component, red yeast rice, coenzyme Q10, L-carnitine, and ascorbic acid.

In another aspect, the invention features the beverage being administered to a human subject twice daily in two servings.

In another aspect, the invention features the two daily servings being stored in a single container.

In another aspect, the invention features each serving of the beverage including about 5 to 100 mg of niacin.

In another aspect, the invention features each serving of the beverage including about 500 to 1,500 mg of the phytosterol component.

In another aspect, the invention features each serving of the beverage including about 300 to 1,500 mg of red yeast rice.

In another aspect, the invention features each serving of the beverage including about 50 to 250 mg of L-carnitine.

In another aspect, the invention features each serving of the beverage including about 300 to 1,200 mg of ascorbic acid.

In another aspect, the invention features each serving of the beverage including about 10 to 200 mg of coenzyme Q10.

In another aspect, the invention features each serving of the beverage including less than about 50 mg of niacin.

In another aspect, the invention features the phytosterol component including at least one phytosterol ester.

In another aspect, the invention features the beverage including at least three active ingredients selected from among the following: niacin, a phytosterol component, red yeast rice, coenzyme Q10, L-carnitine, and ascorbic acid.

In another aspect, the invention features the beverage including at least four active ingredients selected from among the following: niacin, a phytosterol component, red yeast rice, coenzyme Q10, L-carnitine, and ascorbic acid.

In another aspect, the invention features the beverage including at least five active ingredients selected from among the following: niacin, a phytosterol component, red yeast rice, coenzyme Q10, L-carnitine, and ascorbic acid.

In another aspect, the invention features the beverage including niacin, a phytosterol component, red yeast rice, coenzyme Q10, L-carnitine, and ascorbic acid.

In another aspect, the invention features the beverage including maltodextrin.

In another aspect, the invention features the beverage including two daily servings. Each serving can include as ingredients water, about 5 to 50 mg of niacin, about 500 to 1,500 mg of at least one phytosterol ester, about 300 to 1,500 mg of red yeast rice, about 10 to 200 mg of coenzyme Q10, about 50 to 250 mg of L-carnitine, and about 300 to 1,200 mg of ascorbic acid.

In another aspect, the invention features two servings of the beverage being packaged in a container.

In another aspect, the invention features the beverage including water, about 50 to 200 mg of niacin, about 1,400 to 4,000 mg of a phytosterol component, about 1,200 to 4,800 mg of red yeast rice, about 20 to 400 mg of coenzyme Q10, about 400 to 2,000 mg of L-carnitine, and about 200 to 4,000 mg of ascorbic acid.

The invention also features a method that includes the step of administering to a subject for at least 7 days a beverage comprising water and at least two active ingredients selected from among the following: niacin, a phytosterol component, red yeast rice, coenzyme Q10, L-carnitine, and ascorbic acid.

In another aspect, the method also features the step of administering the beverage to the subject twice daily.

The invention also features a composition for reducing blood cholesterol. The composition includes niacin, a phytosterol component, red yeast rice, coenzyme Q10, L-carnitine, and ascorbic acid.

Unless otherwise defined, all technical terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents and other references mentioned herein are incorporated by reference in their entirety. In the case of conflict, the present specification, including definitions will control.

### DETAILED DESCRIPTION

The invention provides an aqueous composition of ingredients and dietary supplements to yield a cholesterol lowering drink. The composition is typically contained within a two-serving container such as a can or bottle and includes water and a combination of at least two of niacin, a phytosterol component, red yeast rice, coenzyme Q10, L-carnitine, and ascorbic acid in amount effective to reduce serum cholesterol levels in a subject who drinks the composition on a regular basis (e.g., once a day, twice a day, every two days, or every three days).

The active ingredients are preferably included at a concentration effective to reduce a subject's serum cholesterol by at least 10% (e.g., at least 20, 30, 40, or 50%) when included in combination with each other and administered to the subject on a regular basis (e.g., twice a day or once a day for at least 2, 3, 4, 5, 6, 12, or 24 weeks). In an exemplary embodiment, the composition can be administered twice daily to the subject in the form of and as part of a beverage, e.g., as a nutritional drink.

Niacin lowers cholesterol by inhibiting lipoprotein formation or release in the liver. Phytosterols lower cholesterol by competing for absorption in the intestines. Red Yeast Rice is a dietary supplement and coloring agent that has been used in Chinese food and medicines for centuries. It decreases synthesis and absorption of cholesterol, produces red coloration which stimulates appetite, compliance, and metabolism. On a cellular level, L-carnitine and coenzyme Q10 enhance transport of fatty acids to the mitochondria and enhance the burning of fatty acids respectively. L-carnitine can also cause a mild sense of euphoria, a beneficial effect that may result in greater compliances with the preparation containing it. Ascorbic acid changes the constitution of bile to decrease cholesterol absorption and inhibit the HMG-CoA reductase pathway, the rate-limiting step in cholesterol biosynthesis.

In an exemplary embodiment, the phytosterol component can include one or more phytosterol esters.

In one embodiment, the beverage can include 6.9 g of a composition in an 8-ounce serving. The beverage can be packaged in a bottle or other container that contains two servings. The composition can include the following ingredients in each 8-ounce serving: about 12.5 mg of niacin (niacinamide), about 500 mg of vitamin C (ascorbic acid), about 150 mg of L-carnitine (L-carnitine-L-tartrate), about 25 mg of coenzyme Q10, about 660 mg of phytosterol esters, and q.s. maltodextrin. Servings of the beverage can be consumed twice daily, for example, one serving in the morning and one serving at night.

In another embodiment, the beverage can include 8.2 g of a composition in an 8-ounce serving. The beverage can be packaged in a bottle or other container that contains two servings. The composition can include the following ingredients in each 8-ounce serving: about 600 mg of red yeast rice powder, about 12.5 mg of niacin (niacinamide), about 500 mg of vitamin C (ascorbic acid), about 150 mg of L-carnitine (L-carnitine-L-tartrate), about 25 mg of coenzyme Q10, about 660 mg of phytosterol esters, and q.s. maltodextrin. Servings of the beverage can be consumed twice daily, for example, one serving in the morning and one serving at night.

Although 8-ounce servings are described herein, the serving size can be different as long as the amounts of each ingredient remain consistent. For example, each serving can be 6, 7, 7.5, 8.5, 9, 10, 12, 13, 14, or 16 ounces.

Each serving of the composition can include niacin in the amounts of about 4.5, 5, 6, 6.1, 6.5, 6.6, 6.9, 7, 7.5, 8, 9, 9.9, 10, 10.1, 10.5, 11, 11.5, 11.9, 12, 12.1, 12.5, 12.6, 12.9, 13, 15, 19, 20, 25, 30, 40, 50, 60, 70, 75, 80, 90, 99, 100, 101, or 110 mg or more. In exemplary embodiments, each serving of the composition can contain less than about 50 mg of niacin. In one exemplary embodiment, each serving of the composition can contain about 12.5 mg of niacin.

Each serving of the composition can include phytosterol esters in the amounts of about 300, 400, 500, 550, 600, 625, 640, 649, 651, 660, 675, 700, 800, 900, 1,000, 1,250, 1,320, 1,500, 1,800, 1,900, 1,950, 1,999, 2,000, 2,001, 2,050, or 2,100 mg or more. In exemplary embodiments, each serving of the composition can contain about 660 mg of phytosterol esters.

Each serving of the composition can include coenzyme Q in the amounts of about 8, 9, 10, 13, 15, 17, 19, 20, 22.5, 24, 24.5, 24.9, 25, 25.1, 25.5, 26, 27, 27.5, 28, 29, 30, 35, 40, 45, 50, 55, 75, 90, 100, 150, 200, 250, or 300 mg or more. In exemplary embodiments, each serving of the composition can contain about 25 mg of coenzyme Q.

Each serving of the composition can include L-carnitine in the amounts of about 90, 95, 100, 110, 115, 125, 135, 140, 145, 149, 149.1, 149.9, 150, 150.1, 150.5, 151, 153, 155, 160, 170, 175, 180, 190, 200, 250, 300, 400, or 500 mg or more. In exemplary embodiments, each serving of the composition can contain about 75-150 mg of L-carnitine. In one exemplary embodiment, each serving of the composition can contain about 150 mg of L-carnitine.

Each serving of the composition can include ascorbic acid in the amounts of about 50, 100, 200, 250, 300, 400, 450, 475, 490, 499, 499.1, 500, 500.1, 500.5, 501, 510, 520, 535, 550, 600, 625, 640, 649, 651, 660, 675, 700, 800, 900, 1,000, 1,500, or 2,000 mg or more. In exemplary embodiments, each serving of the composition can contain about 500 mg of ascorbic acid.

Each serving of the composition can include red yeast rice in the amounts of about 300, 400, 500, 550, 575, 590, 595, 599, 599.1, 599.9, 600, 600.1, 600.5, 601, 610, 620, 625, 640, 650, 660, 675, 700, 800, 900, 1,000, 1,250, 1,500, 1,800, 1,900, 1,950, 1,999, 2,000, 2,001, 2,050, 2,100, or 2,400 mg or more. In exemplary embodiments, each serving of the composition can contain about 600 mg of red yeast rice.

The composition can also feature maltodextrin making up the remainder of any 6.9 g, 8.2 g, or other amount of the composition premixture before the addition of water to create the beverage. The beverage may also contain artificial or natural flavorings and colorings.

### EXAMPLE 1

A beverage for administration twice daily to a human subject can include two servings stored in a container such as, for example, a bottle. Each serving of the beverage can include water (100-500 ml) and 6.9 g of the composition. The composition can include as ingredients phytosterol esters (about 660 mg), niacin (less than about 50 mg), coenzyme Q10 (about 25 mg), ascorbic acid (about 500 mg), L-carnitine (about 75-150 mg), and maltodextrin (q.s.).

### EXAMPLE 2

A beverage for administration twice daily can include two servings stored in a container such as, for example, a bottle or can. Each serving of the beverage can include water (about 100 to 500 ml) and 8.2 g of the composition. As ingredients, the composition can feature phytosterol esters (about 660 mg), niacin (less than about 50 mg), red yeast rice (about 600 mg), coenzyme Q10 (about 25 mg), ascorbic acid (about 500 mg), L-carnitine (about 75-150 mg), and maltodextrin (q.s.).

In an exemplary method of the invention, the composition can be administered to a human subject at least twice daily in the form of a beverage or nutritional drink. The two daily doses of the beverage can be contained within a single container such as, for example, a bottle or can. In another embodiment, each serving of the beverage can be packaged in a separate bottle or container. The beverage can be administered twice daily for at least 7 days. The beverage can include water and at least two active ingredients selected from among the following: niacin, a phytosterol component, red yeast rice, coenzyme Q10, L-carnitine, and ascorbic acid.

In another method of the invention, the composition may be administered at an interval different than twice daily, e.g., once, three times, or four times daily.

In still another method of the invention, the composition can be administered to a human subject to reduce the subject's blood cholesterol levels.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A nutritional beverage comprising water and at least two active ingredients selected from the group consisting of niacin, a phytosterol component, red yeast rice, coenzyme Q10, L-carnitine, and ascorbic acid.

2. The nutritional beverage of claim 1, wherein the beverage is administered to a human subject twice daily in two servings.

3. The nutritional beverage of claim 2, wherein the two daily servings are stored in a single container.

4. The nutritional beverage of claim 2, wherein each serving of the beverage comprises about 5 to 100 mg of niacin.

5. The nutritional beverage of claim 2, wherein each serving of the beverage comprises about 500 to 1,500 mg of the phytosterol component.

6. The nutritional beverage of claim 2, wherein each serving of the beverage comprises about 300 to 1,500 mg of red yeast rice.

7. The nutritional beverage of claim 2, wherein each serving of the beverage comprises about 50 to 250 mg of L-carnitine.

8. The nutritional beverage of claim 2, wherein each serving of the beverage comprises about 300 to 1,200 mg of ascorbic acid.

9. The nutritional beverage of claim 2, wherein each serving of the beverage comprises about 10 to 200 mg of coenzyme Q10.

10. The nutritional beverage of claim 2, wherein each serving of the beverage comprises less than about 50 mg of niacin.

11. The nutritional beverage of claim 1, wherein the phytosterol component comprises at least one phytosterol ester.

12. The nutritional beverage of claim 1, wherein the beverage comprises at least three active ingredients selected from the group consisting of niacin, a phytosterol component, red yeast rice, coenzyme Q10, L-carnitine, and ascorbic acid.

13. The nutritional beverage of claim 1, wherein the beverage comprises at least four active ingredients selected from the group consisting of niacin, a phytosterol component, red yeast rice, coenzyme Q10, L-carnitine, and ascorbic acid.

14. The nutritional beverage of claim 1, wherein the beverage comprises at least five active ingredients selected from the group consisting of niacin, a phytosterol component, red yeast rice, coenzyme Q10, L-carnitine, and ascorbic acid.

15. The nutritional beverage of claim 1, wherein the beverage comprises niacin, a phytosterol component, red yeast rice, coenzyme Q10, L-carnitine, and ascorbic acid.

16. The nutritional beverage of claim 1, wherein the beverage comprises maltodextrin.

17. The nutritional beverage of claim 11, wherein the beverage comprises two daily servings, each serving comprising water, about 5 to 50 mg of niacin, about 500 to 1,500 mg of at least one phytosterol ester, about 300 to 1,500 mg of red yeast rice, about 10 to 200 mg of coenzyme Q10, about 50 to 250 mg of L-carnitine, and about 300 to 1,200 mg of ascorbic acid.

18. A method comprising the step of administering to a subject for at least 7 days a beverage comprising water and at least two active ingredients selected from the group consisting of niacin, a phytosterol component, red yeast rice, coenzyme Q10, L-carnitine, and ascorbic acid.

19. The method of claim 18, wherein the beverage is administered to the subject twice daily.

20. A composition for reducing blood cholesterol comprising: niacin, a phytosterol component, red yeast rice, coenzyme Q10, L-carnitine, and ascorbic acid.
